(19) United States
(12) Patent Application Publication
(10) Pub. No.: US 2025/0249283 A1
(43) Pub. Date: Aug. 7, 2025

(54) FIXATION ELEMENT FOR BRACHYTHERAPY APPLICATOR

(71) Applicant: ELEKTA LIMITED, Crawley Sussex (GB)

(72) Inventor: Jan Willem Van Manen, Veenendaal (NL)

(21) Appl. No.: 18/853,044

(22) PCT Filed: Mar. 31, 2023
(86) PCT No.: PCT/EP2023/058577

§ 371 Date: Sep. 30, 2024

(30) Foreign Application Priority Data
Mar. 31, 2022 (GB) 2204730.2
Oct. 5, 2022 (GB) 2214650.0

(51) Int. Cl. A61N 5/10

(52) U.S. Cl. CPC A61N 5/1014

(57) ABSTRACT

Disclosed herein is a fixation element for an applicator for use in brachytherapy. The fixation element comprises a first component comprising a camming portion and a lever portion; and a second component comprising a securement portion forming an opening for receiving the applicator. The securement portion comprises an abutment portion, and a first component receiving portion. Wherein, in a first position, the first component is received in the first component receiving portion, and wherein the fixation element is configured to transition from the first position to a second position as the lever portion is actuated. The actuation of the lever portion causing the camming portion to exert a force against the abutment portion, and as the force is exerted against the abutment portion, the opening formed by the securement portion constricts.

This disclosure relates generally to a fixation element for a brachytherapy applicator, and to a brachytherapy applicator comprising a fixation element. In particular, this disclosure relates to a fixation element for an intracavitary brachytherapy applicator, and to an intracavitary brachytherapy applicator comprising a fixation element.

### BACKGROUND

Radiotherapy can be described as the use of ionising radiation to damage or destroy unhealthy cells in both humans and animals. The ionising radiation may be directed to tumours on the surface of the skin or deep inside the body. Common forms of ionising radiation include X-rays and charged particles. Brachytherapy is a form of internal radiotherapy, in which a radioactive source is inserted or positioned within the body, in or in close proximity to the tumour. Brachytherapy can provide a targeted approach to radiotherapy, as the ionising radiation can be applied more closely to a target region (e.g. the tumour). The proximity of the radiation source to the target region can reduce the amount of radiation delivered to healthy tissue and organs, for example by eliminating the need for radiation to penetrate through skin and muscle before reaching the target region.

The internal radiation source may be positioned based on the location and/or type of cancer being treated. There are two types of brachytherapy—intracavitary and interstitial. Interstitial brachytherapy involves the placement, or implantation, of a radiation source within body tissue, in close proximity to the target region. The radiation source may be either temporarily or permanently implanted. Intracavitary brachytherapy involves the insertion of an applicator into a body cavity, such as a patient's oesophagus, rectum or vagina.

During a treatment using intracavitary brachytherapy, the applicator may be inserted into the patient's body cavity and held in place to ensure that the radiation source does not move during the treatment. For example, cancers such as rectal cancer can be treated using endorectal brachytherapy, in which a rectal applicator is inserted into a patient's rectum. A radiation source is provided via the applicator. However, upon insertion of the rectal applicator, the muscles of the rectum naturally attempt to expel the applicator, making it difficult to keep the rectal applicator in place during the treatment. For this reason, a fixation element such as a clamp is typically used to fix the position of the applicator in the patient's rectum relative to the patient, to prevent the expulsion of the applicator.

Current fixation elements are made up of several metal components, which need to be assembled, and subsequently disassembled, using additional tooling, such as screws and screwdrivers, and many alternatives are rigidly fixed by, e.g. being glued. Such assembly is time consuming and cumbersome, as it requires at least two hands to secure, and is uncomfortable for the patient. This prolongs the treatment, and therefore the discomfort, experienced by the patient.

Moreover, current assemblies are difficult to clean and sterilise, and the metal components render the fixation elements unsuitable for use with magnetic resonance equipment. MRI can be used during treatment to check the position of the radiation source for treatment planning and/or for optimising radiation dose delivery, but these advantages are unavailable if the equipment used in the brachytherapy treatment is not MR compatible.

The present invention seeks to address these and other issues encountered in the prior art.

### SUMMARY

Aspects and features of the present disclosure are described in the accompanying claims.

According to an aspect, the present disclosure provides a fixation element for an applicator for use in brachytherapy, the fixation element comprising: a first component comprising a camming portion and a lever portion; and a second component comprising a securement portion forming an opening for receiving the applicator, the securement portion comprising an abutment portion, and a first component receiving portion. In a first position, the first component is received in the first component receiving portion. The fixation element is configured to transition from the first position to a second position as the lever portion is actuated, the actuation of the lever portion causing the camming portion to exert a force against the abutment portion. As the force is exerted against the abutment portion, the opening formed by the securement portion constricts.

In some implementations, the fixation element may be resiliently deformable between the first position and the second position.

In some implementations, the first component may further comprise at least one extending portion protruding from the camming portion, and the first component receiving portion may comprise a respective at least one groove arranged to receive the at least one extending portion.

In some implementations, the at least one extending portion may be oblong.

In some implementations, the first component receiving portion may comprise a respective at least one recess coupled to the at least one groove, the respective at least one recess arranged such that, in the first position, the extending portion is removable from the at least one recess via the at least one groove, and in the second position, the extending portion is retained in the at least one recess.

In some implementations, the second component may comprise a fastening portion for securing a position of the fixation element with respect to a patient.

In some implementations, the second component may comprise an attachment portion for securing a position of the fixation element with respect to a patient support surface.

In some implementations, the fixation element may be for receiving a rectal applicator.

In some implementations, the camming portion may be asymmetric.

In some implementations, the second component may be resiliently compressible.

In some implementations, the fixation element may be configured to transition from the first position to the second position through a pivoting motion of the first component.

In some implementations, the pivoting motion of the first component may cause a rotation of the first component.

In some implementations, the actuation of the lever portion may rotate the camming portion, the rotation of the camming portion causing the camming portion to exert a force against the abutment portion.

In some implementations, the fixation element may be configured to transition from the first position to the second position by a deformation of the camming portion.

In some implementations, the deformation of the camming portion may be a compression of the camming portion.

According to another aspect, the present disclosure provides an applicator for brachytherapy, comprising the fixation element.

In some implementations, the applicator may comprise a substantially cylindrical body on which the fixation element may be slidably mounted when the fixation element is in the first position, and restrained on said cylindrical body when in the second position.

In some implementations, the opening may be substantially circular.

In some implementations, the applicator may be a rectal applicator.

### BRIEF DESCRIPTION OF THE DRAWINGS

Specific embodiments are now described, by way of example, with reference to the drawings, in which:

FIG. 1 depicts an applicator for brachytherapy positioned within a patient's body cavity according to the present disclosure;

FIG. 2 depicts an applicator for brachytherapy according to the present disclosure;

FIG. 3a depicts a fixation element for an applicator according to the present disclosure;

FIGS. 3b and 3c depict components of a fixation element for an applicator according to the present disclosure;

FIG. 4 shows a first component of a fixation element according to the present disclosure;

FIG. 5 shows a second component of a fixation element according to the present disclosure;

FIGS. 6a-6c illustrate an assembly of a fixation element according to the present disclosure;

FIGS. 7a and 7b show a fixation element in a first position according to the present disclosure;

FIGS. 8a and 8b show a fixation element in an intermediary position according to the present disclosure;

FIGS. 9a and 9b show a fixation element in a second position according to the present disclosure; and

FIGS. 10a and 10b illustrate examples of a locking mechanism according to the present disclosure.

### LIST OF REFERENCE SIGNS

10 Applicator
20 Patient's body cavity
30 Fixation element
40 First component of the fixation element
42 Lever portion
44 Camming portion
45 Camming surface
46 Extending portion
50 Second component of the fixation element
51 Securement portion
52 Opening
53 Abutment portion
54 First component receiving portion
55 Groove
56 Recess
57 Fastening portion
58 Attachment portion

### DETAILED DESCRIPTION

Aspects of the disclosure will be described below. In overview, and without limitation, the present application relates to a fixation element for an applicator for use in brachytherapy, as well as the combined applicator and fixation element. The fixation element comprises two components, which can be easily and quickly assembled and disassembled. The fixation element may be opened and/or closed without the use of any additional tools, making the set-up and dismantling of the fixation element quicker and easier than current fixation elements. The components may also be more easily cleaned and sterilised. As separate (e.g. metal) fasteners are not necessary, the fixation element may be made in a material which is MR-compatible.

Throughout the detailed description, reference is made to a rectal applicator. However, it is to be understood that the disclosure is not limited thereto and can be applicable to other brachytherapy applicators where it may be necessary to restrain the applicator at a predefined position inserted into a patient.

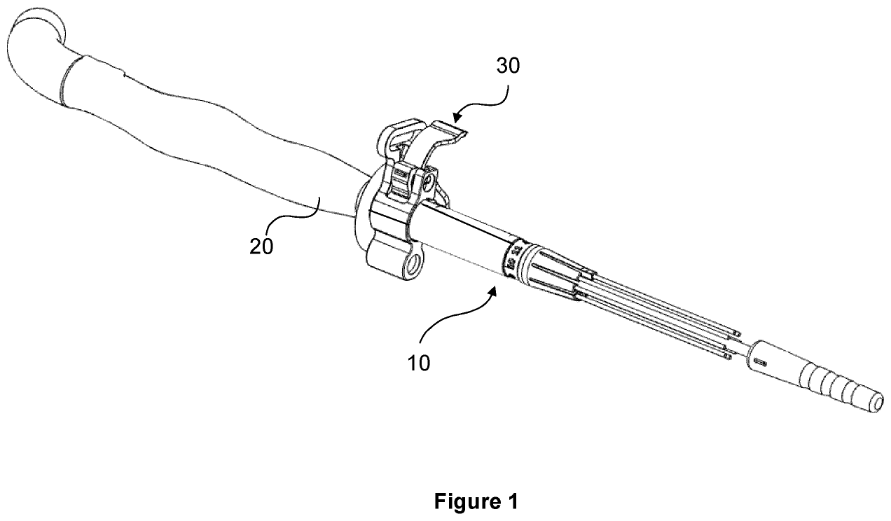

FIG. 1 depicts an applicator 10 for brachytherapy positioned within a patient's body cavity 20 according to the present disclosure.

The applicator 10 may be positioned within the patient's body cavity 20 using a fixation element 30. During a brachytherapy treatment, the applicator 10 is inserted into the body cavity 20. Once the applicator is in position, the fixation element 30 may be used to keep the applicator 10 in position relative to the patient. For example, the fixation element 30 may be securely clamped to the applicator 10 such that the fixation element 30 abuts against the opening of the patient's body cavity 20, e.g. the rectum. Once the fixation element 30 is in place, additional fixation means such as bandages or tape may be attached to the fixation element 30 to secure the applicator 10 in position, relative to the patient's anatomy.

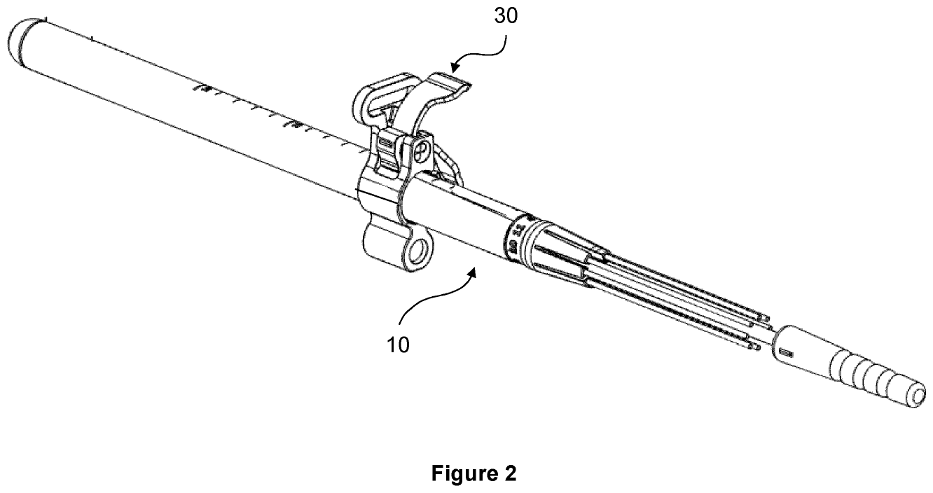

Whilst FIG. 1 illustrates the applicator 10 inserted into the patient's body cavity 20, FIG. 2 depicts the applicator 10 and the fixation element 30 without the patient's body cavity. In some embodiments, the fixation element 30 may be a component of the applicator 10. In other words, the applicator 10 may comprise the fixation element 30. The applicator 10 may be an intracavitary applicator, such as a rectal applicator. In some embodiments, the fixation element 30 may be a separate component, and may be retrofitted to existing applicators. For example, the shape of the opening may be profiled to the external shape of an existing applicator. The fixation element 30 may be configured to removably attach to the applicator 10, for example at a region of the applicator 10 which remains outside of the patient's body cavity 20 during use.

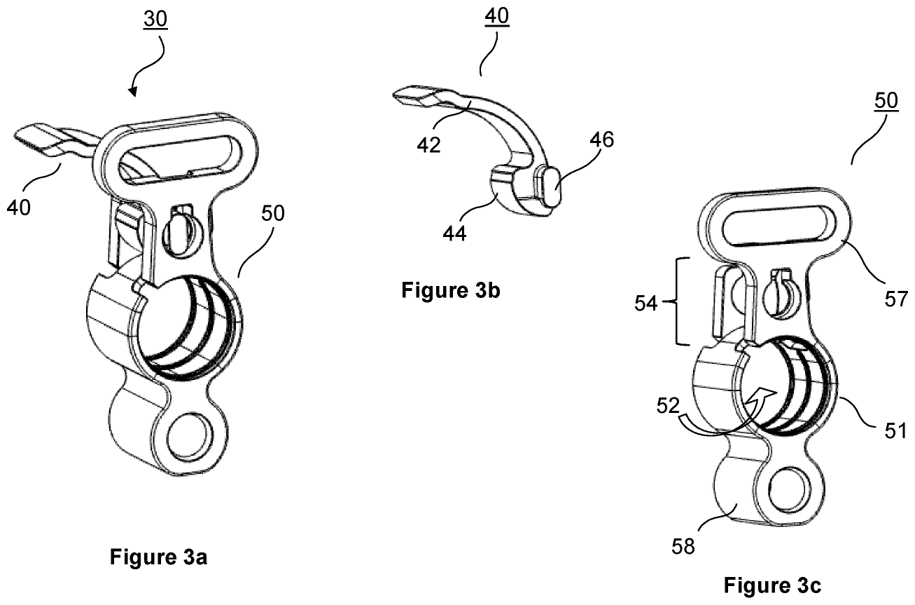

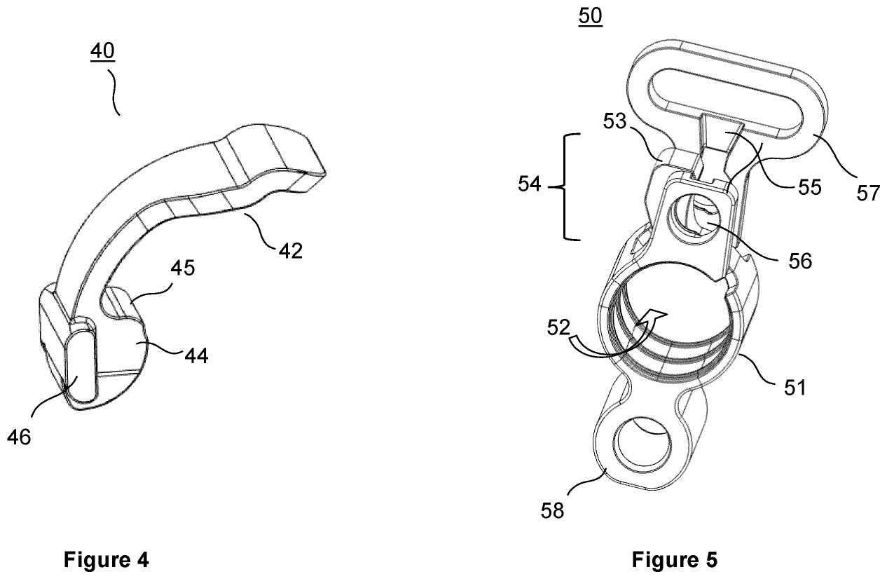

FIG. 3a depicts a fixation element 30 for an applicator 10 according to the present disclosure. The applicator 10 may be a rectal applicator. The fixation element 30 comprises a first component 40 and a second component 50. The fixation element 30 is illustrated assembled in FIG. 3a, in which the first component 40 is removably inserted in the second component 50. The fixation element 30 is illustrated disassembled in FIGS. 3b and 3c. The first component 40 will be further described with reference to FIG. 4, whilst the second component 50 will be described with reference to FIG. 5.

Referring now to FIG. 4, the first component 40 of the fixation element 30 comprises a lever portion 42 and a camming portion 44. In some embodiments, the camming portion 44 may comprise at least one extending portion 46. For example, an extending portion 46 may be disposed on each side of the camming portion 44. The lever portion 42 may be monolithic with the camming portion 44. For example, the first component 40 may be rigid. In other words, movement of the lever portion 42 effects movement of the camming portion 44.

The camming portion 44 may have two flat, or substantially flat, sides, from which at least one extending portion 46 may protrude. The camming portion 44 may comprise a camming surface 45. The two sides of the camming portion 44 may be, for example, orthogonal or substantially orthogonal to the camming surface 45. The camming surface 45 may be arranged to contact an abutment portion 53 of the second component 50 (described with reference to FIG. 5) upon assembly of the fixation element 30 and during use. The camming portion 44 may be asymmetric. For example, the camming portion 44 may be wider at one end than at the other end. The camming portion 44 having an asymmetric shape may be particularly suited to providing a force upon rotation of the first component 40.

The first component 40 may comprise a single extending portion 46, e.g. protruding from one side of the camming portion 44. In some embodiments, the first component 40 may comprise two extending portions 46, e.g. protruding from each side of the camming portion 44. For example, the two extending portions 46 may be monolithic, e.g. the extending portions 46 may form a single piece arranged to protrude from both sides of the camming portion 44. In some embodiments, the extending portion(s) 46 may be removably attached to the camming portion 44, for example to facilitate easier cleaning and maintenance of the fixation element 30. The at least one extending portion 46 may be elongated, such as oblong, elliptical or rectangular in shape. In other words, a cross-section of the at least one extending portion 46 may be oblong, elliptical or rectangular. The at least one extending portion 46 may be described as having a width and a length, in which the length is greater than the width.

The first component 40 may be made of a non-metallic material. The material of the first component 40 may be a magnetic resonance (MR)-compatible material. For example, the first component 40 may be made of a plastic, e.g. polyphenylsulfone (PPSU). However, whilst non metallic components may be preferable to allow operation compatible with MRI, the components used in the fixation element may incorporate or comprise a metallic element such as stainless steel where non MRI use is anticipated.

In some implementations, the camming portion 44 may be made of a resiliently compressible material, for example a resiliently compressible plastic such as PPSU or silicone coated PPSU. In particular, in some implementations, the first component 40 may be configured to apply a force, or pressure, to the securement portion 51 of the second component by compressing as the lever portion 42 is pressed. In some implementations, the first component 40 may be made of a resiliently compressible material.

In some implementations, an extending portion may be additionally or alternatively disposed on the securement portion 51. For example, the lever portion 42 may be configured to be caught, or locked, by an extending portion such as a clasp, latch or catch.

Referring now to FIG. 5, the second component 50 of the fixation element 30 comprises a securement portion 51 forming an opening 52, an abutment portion 53 and a first component receiving portion 54. The securement portion 51 may form an opening 52 configured to receive an applicator, such as applicator 10 which may be a rectal applicator. That is, the opening 52 may have a larger diameter than the applicator 10. The opening 52 may have a larger diameter when the fixation element 30 is in an open position, and may have a smaller diameter when the fixation element 30 is in a closed position. That is, transitioning from an open position to a closed position of the fixation element 30 may reduce the diameter of the opening 52, e.g. tightening the opening 52. The second component 50 of the fixation element 30 may, preferably but not exclusively, be made of a non-metallic material. In some embodiments, the second component 50 of the fixation element 30 may be made of a resiliently compressible material, such as a resiliently compressible plastic (e.g. PPSU).

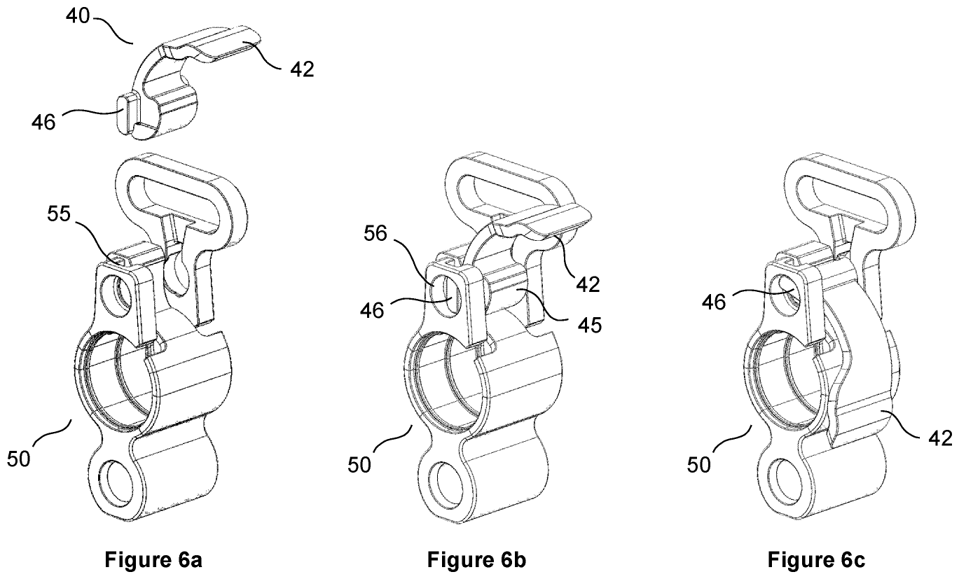

The first component receiving portion 54 is configured to receive the first component 40 of the fixation element 30. The first component receiving portion 54 may comprise at least one groove 55, arranged to receive the at least one extending portion 46 of the first component 40. That is, the width of the at least one groove 55 may be large enough to accommodate the at least one extending portion 46. The width of the at least one groove 55 may be less than the length of the at least one extending portion 46. The purpose of the at least one groove 55 having a width between the width of the at least one extending portion 46 and the length of the at least one extending portion 46 will be described with reference to FIGS. 6a-6c. In some embodiments, the at least one groove 55 may be tapered, as illustrated in FIG. 5, although this is not required. In such embodiments, the narrowest part of the at least one groove 55 may have a width greater than the width of the at least one extending portion 46 and less than the length of the at least one extending portion 46.

The first component receiving portion 54 may comprise at least one recess 56. The at least one recess 56 may be circular. The at least one groove 55 may extend to the respective at least one recess 56. In some embodiments, the at least one recess 56 may be a hole (as shown in FIG. 5). In other embodiments, the at least one recess 56 may not extend through the full thickness of the material of the first component receiving portion 54, but may have a depth large enough to receive the at least one extending portion 46 of the first component 40. The at least one recess 56 may be dimensioned to allow the at least one extending portion 46 to rotate within the at least one recess 56 when the fixation element 30 is assembled.

The second component 50 of the fixation element 30 comprises an abutment portion 53. The abutment portion 53 is coupled to the securement portion 51 such that, as a force is applied to the abutment portion in a first direction, the opening 52 formed by the securement portion 51 reduces in size, e.g. tightens. The abutment portion 53 is arranged in the first component receiving portion 54 of the second component 50 such that, when the fixation element 30 is assembled, the camming portion 44 of the first component 40 contacts the abutment portion 53. As the lever portion 42 of the first component 40 is pressed (e.g. into a closed, or second, position), the camming portion 44 is arranged to apply a force to the abutment portion 53, causing the securement portion 51 to tighten and reducing the diameter of the opening 52.

In some embodiments, the second component 50 may comprise a fastening portion 57. The fastening portion 57 may be arranged to allow fastening means, such as bandages, to be attached to the fixation element 30. During use, once the fixation element 30 has been securely fastened to the applicator 10, the position of the applicator 10 may be secured relative to the patient's body by attaching bandages, for example, to the fastening portion 57 and to the patient's body (e.g. around the patient's body). The position of the applicator 10, and thus the position of a radioactive source in or on the applicator 10 within the patient's body cavity, may be maintained during treatment.

In some embodiments, the second component 50 may comprise an attachment portion 58. The attachment portion 58 may be arranged to be secured to a patient treatment surface, such as a patient positioning surface, gurney, bed or couch used in radiotherapy. The attachment portion 58 may enable the fixation element 30 to be attached to the patient treatment surface through any known fastening means, including but not limited to a clasp, clamp, one or more screws, bolts, nails, etc., or a locking mechanism.

FIGS. 6a-6c illustrate an assembly of a fixation element according to the present disclosure. In particular, FIG. 6a illustrates the fixation element 30 in a disassembled state, e.g. in which the first component 40 is not inserted into the second component 50. FIG. 6b shows the fixation element 30 in which the first component 40 and the second component 50 are assembled, and in which the fixation element 30 is in a first, e.g. open, position. FIG. 6b shows the fixation element 30 in a second, e.g. closed position.

The fixation element 30 may be assembled by inserting the first component 40 into the second component 50. That is, the first component 40 may be inserted into the first component receiving portion 54 of the second component 50. The first component 40 may be inserted by inserting at least one extending portion 46 into the at least one groove 55. In order to insert the at least one extending portion 46 into the at least one groove 55, the first component 40 may be orientated such that the at least one extending portion 46 fits through the at least one groove 55.

During use, the second component 50 of the fixation element 30 may be positioned on the applicator 10 prior to assembly of the fixation element 30. For example, the second component 50 may be provided on the applicator 10 or may be slid onto the applicator 10. That is, an applicator 10 may comprise a fixation component 30. The second component 50 may be removably coupled to the applicator 10.

Upon insertion of the first component 40 into the second component 50, the at least one extending portion 46 may be disposed within a respective at least one recess 56. In the first (e.g. open) position, the lever portion 42 may be in a raised, e.g. open, position and the at least one extending portion 46 may be in a vertical position as shown. In the first (e.g. open) position, the camming portion 44 of the first component 40 may be arranged to be in contact with the securement portion 51 of the second component 50.

During use, the first component 40 may be inserted into the second component 50 of the fixation element 30. Inserting the first component 40 into the second component 50, e.g. using the at least one groove 55, may be done using one hand, making assembly easy and quick. A quick assembly of the fixation element 30 may reduce the time required for treatment set-up and may reduce the discomfort experienced by the patient, as no additional tools or components are required.

FIG. 6b illustrates the fixation element 30 in the first, e.g. open, position. In this position, the lever portion 42 is raised, the at least one extending portion 46 may be in a vertical position (e.g. parallel to the groove 55). As the lever portion 42 of the first component 40 is pressed, e.g. towards the applicator 10, the camming portion 44 abuts against the abutment portion 53 of the second component 50 of the fixation element. The force against the abutment portion 53 applied by the camming portion 44 causes the opening 52 to become smaller (e.g. makes the diameter of the opening 52 smaller), thereby tightening the fixation element 30 onto the applicator 10.

In some implementations, the first component 40 may be configured to rotate about a pivot point, e.g. to transition from the first position to the second position through a pivoting motion of the first component. In some implementations, the pivoting motion of the first component causes a rotation of the first component. In some implementations, the actuation of the lever portion 42 may cause the camming portion 44 to rotate, such that a force is exerted against the abutment portion. That is, the first component 40 may be substantially rigid, such that a pivoting motion of the first component 40 causes the camming portion 44 to rotate and apply a force or pressure against the abutment portion 53 of the second component 50 when the fixation element 30 transitions from the first position to the second position. The fixation element 30 may be resiliently deformable. For example, when the fixation element 30 transitions from the second position to the first position, e.g. to open the fixation element 30, each of the first component 40 and the second component 50 may be resiliently deformable. In other words, upon opening the fixation element 30, the opening of the second component 50 may return to its original shape and size.

In some implementations, the pivoting motion of the first component may cause a lowering or a depression of the first component. The lowering or depression of the first component may be substantially linear. In some implementations, the actuation of the lever portion 42 may cause a lowering or depression of the camming portion 44, such that a force is exerted against the abutment portion. In other words, a lowering or depression of the camming portion may cause the camming portion to press against the abutment portion of the second component. That is, the first component 40 may be substantially rigid, such that a pivoting motion of the first component 40 causes a lowering of the camming portion 44, wherein the camming portion applies a force or pressure against the abutment portion 53 of the second component 50 when the fixation element 30 transitions from the first position to the second position. The fixation element 30 may be resiliently deformable. For example, when the fixation element 30 transitions from the second position to the first position, e.g. to open the fixation element 30, each of the first component 40 and the second component 50 may be resiliently deformable. In other words, upon opening the fixation element 30 (e.g. by releasing the lever portion 42), the opening of the second component 50 may return to its original shape and size.

The first component 40 and/or the camming portion 44 may be resiliently compressible. In some implementations, actuation of the lever portion 42 may cause the camming portion 44 to resiliently compress, exerting a force or pressure on the securement portion 51. The force or pressure may be exerted on the abutment portion 53, which may be a portion of the securement portion 51. The force or pressure may be exerted by the camming portion 44 on the abutment portion 53, or the securement portion 51, by actuating the lever portion 42 and/or by a pivoting motion of the first component 40.

FIG. 6c illustrates the fixation element 30 in a second, e.g. closed and/or locked, position. In the second position, the lever portion 42 may be depressed, e.g. fully depressed, against the securement portion 51. In some implementations, pressing the lever portion 42 may cause the camming portion 44, and therefore the at least one extending portion 46, to rotate, thereby applying a force against the abutment portion 53. In some implementations, pressing the lever portion 42 may cause the camming portion 44 to resiliently compress, thereby applying a force against the abutment portion 53 or, more generally, the securement portion 51. In the second position, the at least one extending portion 46 may be in a non-vertical, e.g. a horizontal or substantially horizontal, position. That is, in the second position, the at least one extending portion 46 may be angled such that the at least one extending portion 46 does not fit through the at least one groove 55 without rotation. That is, the at least one groove 55 having a width which is less than the length of the at least one extending portion 46 can prevent the first component 40 from unintentionally decoupling from the second component 50. That is, the at least one groove 55 and the at least one extending portion 46 may form a locking system.

In the second position, the fixation element 30 may be tightened, and therefore removably attached, to the applicator 10. After the treatment is complete, the fixation element 30 may be disassembled by lifting the lever portion 42. As the lever portion 42 is lifted, the pressure (e.g. force) against the abutment portion 53 may be eased, allowing the opening 52 to increase in diameter. This enables the fixation element 30 to be moved along the applicator 10 and/or removed from the applicator 10. That is, disassembly of the fixation element 30 may be performed by transitioning the fixation element 30 from the second position (illustrated in FIG. 6c) to the first position (illustrated in FIG. 6b).

The first component 40 of the fixation element 30 may be removed from the second component 50 of the fixation element 30 easily and quickly, reducing the discomfort experienced by the patient. For example, no additional tools are required for the disassembly. The healthcare provider, or user assembling or disassembling the fixation element 30, may assemble and/or disassemble the fixation element 30 with one hand.

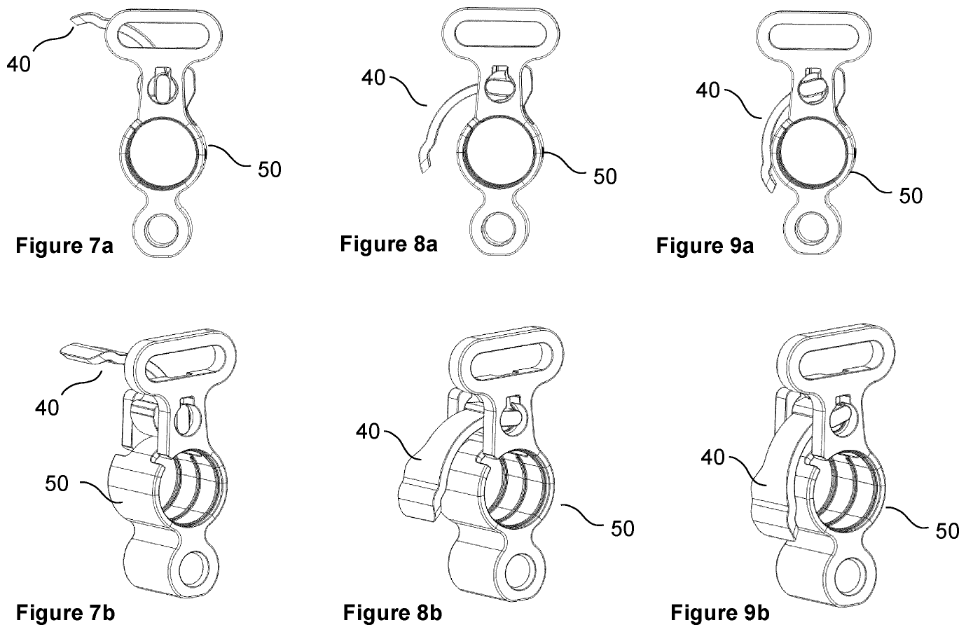

FIGS. 7 through 9 illustrate the fixation element through different positions. FIGS. 7a and 7b show the fixation element 30 in a first position, FIGS. 9a and 9b show the fixation element 30 in a second position, and FIGS. 8a and 8b show the fixation element 30 in an intermediary position between the first and second positions. The first position may be an open position, e.g. a position in which the fixation element 30 is movable, slidably displaceable along the applicator, and/or repositionable. The second position may be a closed position, e.g. a position in which the fixation element 30 is secured to the applicator 10. That is, in the second position, the fixation element 30 may not be movable along the applicator 10. For example, the fixation element 30 may be tightened around the applicator 10 to prevent movement of the fixation element 30 along the applicator 10.

Referring first to FIGS. 7a and 7b, in the first position, the lever portion 42 of the first component 40 may be in a raised position, e.g. away from the securement portion 51 of the second component 50. In the open position, as can be seen in FIG. 7b, the camming surface 45 of the camming portion 44 does not apply a force against the abutment portion 53 of the second component 50 of the fixation element 30.

In the first position, the first component 40 may be separated (e.g. removed or decoupled) from the second component 50 of the fixation element. The first position may be considered an open or unlocked position. In the first position, the opening 52 may be large enough to move along the applicator 10. That is, the fixation element 30 is in the first position while the fixation element 30 is being positioned along the applicator 10. As will be described later, in the first position, the opening 52 has a larger diameter than in the second position (illustrated in FIGS. 9a and 9b).

In some embodiments, the at least one extending portion 46 of the camming portion 44 may be aligned with a respective at least one groove 55 of the second component 50. For example, the at least one extending portion 46 and the at least one groove 55 may be axially aligned. Alignment of the at least one groove 55 and the at least one extending portion 46 allows for the first component 40 of the fixation element 30 to be easily removed and/or inserted into the second component 50 of the fixation element 30.

Compressing, or depressing, the lever portion 42 can transition the fixation element 30 from the first position to the second position, via an intermediary position. As the lever portion 42 is moved toward the securement portion 51, the camming portion 44 is forced against the abutment portion 53.

In some embodiments, the at least one extending portion 46 of the camming portion 44 may act as a pivot point. As the lever portion 42 is depressed, the first component 40 rotates about the pivot point (e.g. the at least one extending portion 46 within the respective at least one recess 56), which causes the camming portion 44 to apply a force against the abutment portion 53 of the second component 50.

In some embodiments, the fixation element 30 (e.g. at least one of the first component 40 and the second component 50) may be resiliently deformable between the first position and the second position.

In some embodiments, the camming portion 44 of the first component 40 may be made of a resiliently and/or elastically deformable material. As the lever portion 42 is depressed, the camming portion 44 deforms and applies a force against the abutment portion 53 of the second component 50. In some implementations, the abutment portion 53 may be positioned on the securement portion 51 to abut against the camming portion 44 of the first component 40 when the fixation element 30 is in the first position. That is, the force against the securement portion 51 (e.g. the abutment portion 53) may be provided by deforming the camming portion 44 when the lever portion 42 is lowered. In some embodiments, the first component 40 may be made of a resiliently deformable material. In other embodiments, a portion of the first component 40 may be made of a resiliently deformable material while other portions of the first component 40 may be made of a rigid material. For example, the camming portion 44 may be made of a resiliently deformable material while the lever portion 42 may be made of a rigid material.

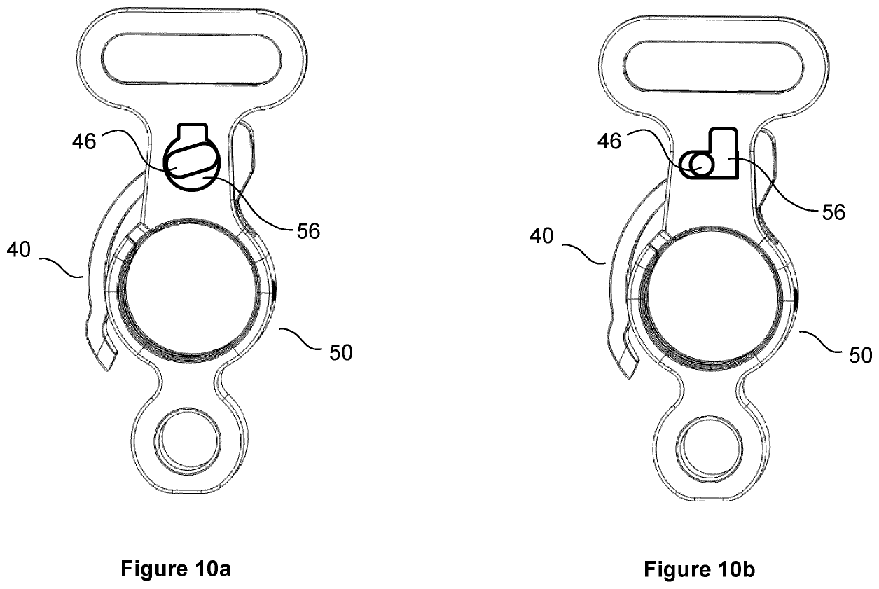

In the second position, the fixation element 30 may be in a closed, locked and/or tightened position in which the fixation element 30 may not be moved along the applicator 10. The fixation element 30 may be transitioned from the first position to the second position by lowering, or depressing, the lever portion 42 of the first component 40. In the second position, the lever portion 42 may be in a lowered position towards, e.g. in proximity to or in contact with, the securement portion 51 of the second component 50. In some embodiments, the lever portion 42 may be shaped to conform with the shape of the securement portion 51. The lever portion 42 conforming with the shape of the securement portion 51 may prevent other materials (such as bandages, cords, tubes etc.) from catching on the lever portion 42 during treatment. The lever portion 42 may be maintained in the second (e.g. closed, or locked) position by means of a locking mechanism. That is, a locking mechanism may be provided which holds (e.g. maintains) the lever portion 42 in the second position. Several examples of such a locking mechanism are elucidated below, although it is to be understood that these are merely exemplary. FIGS. 10a and 10b illustrate examples of a locking mechanism.

In some embodiments, the camming portion 44 may apply a force to the abutment portion 53 as the lever portion 42 is depressed, resulting in a pivoting (e.g. rotational) motion of the first component 40. The first component 40 may be in a locked position relative to the second component 50 by means of a locking mechanism within the first component receiving portion 54. One example of such a locking mechanism may be provided by the at least one extending portion 46, the at least one groove 55 and the at least one recess 56 illustrated in FIGS. 7-9 and in FIG. 10a, although this is merely exemplary. Other locking mechanisms may similarly be employed. As shown in FIG. 10a, the first component 40 may be in a locked position relative to the second component 50 by the rotation of the at least one extending portion 46 within the at least one recess 56. The size, orientation and shape of the at least one extending portion 46 in the second position may prevent the first component 40 from becoming decoupled from the second component 50.

In some embodiments, the camming portion 44 may apply a force to the securement portion 51 (e.g. the abutment portion 53) of the second component as the lever portion 42 is depressed, resulting in a deformation of the camming portion 44. That is, the camming portion 44 may be made of a resiliently and/or elastically deformable material. The lever portion 42 may be kept in place through a locking mechanism such as a latch, clip or the like. For example, the first component receiving portion 54 may comprise one or more protrusions past which the lever portion 42 is lowered. The one or more protrusions may prevent the lever portion 42 from lifting unintentionally. As a further example, the lever portion 42 may be depressed and moved behind or within a rigid clip, hook or clasp. Such a clip, hook, clasp or similar may be disposed in the first component receiving portion 54. FIG. 10b illustrates still a further example. Referring now to FIG. 10b, the at least one recess 56 may have a longitudinal shape, and may be arranged such that the at least one extending portion 46 is moved along the longitudinal at least one recess 56 when the lever portion 42 is depressed. That is, the at least one extending portion 46, the at least one groove 55 and the at least one recess 56 may form the locking mechanism. The locking mechanism may keep the lever portion 42 depressed such that the force or pressure applied to the securement portion 51 (e.g. the abutment portion 53) of the second component 50 by the camming portion 44 of the first component 40 is maintained.

In some embodiments, the fixation element 30 may be provided with an applicator for brachytherapy. The applicator may comprise a substantially cylindrical body on which the fixation element 30 is slidably mounted when the fixation element 30 is in the first position. When the fixation element 30 is in the second position, the fixation element 30 is restrained on the substantially cylindrical body of the applicator. The opening 52 of the fixation element 30 may be substantially circular. The applicator may be a rectal applicator.

The fixation element 30 may be opened, e.g. unlocked, by moving the lever portion 42 from the second (e.g. closed, or locked) position to the first position (e.g. the open position). As the lever portion 42 is moved to the first position, the pressure or force applied to the securement portion 51 (e.g. the abutment portion 53) by the camming portion 44 is reduced (e.g. removed), and the securement portion 51 may be loosened from the applicator 10. That is, upon removal or reduction of the pressure applied by the camming portion 44 to the securement portion 51 (e.g. the abutment portion 53), the fixation element 30 can move along the applicator 10. Upon removal or reduction of the pressure applied by the camming portion 44 to the securement portion 51 (e.g. the abutment portion 53), one or both of the first component 40 and the second component 50 may return to their original shape and size.

From the first position, the first component 40 may be removed or decoupled from the second component 50 easily, by simply lifting the first component 40 out of the first component receiving portion 54 of the second component 50. The user may therefore affix and remove the fixation element 30 to/from the applicator 10 easily, without the need for tools, quickly and with reduced discomfort to the patient.

It is to be understood that the above description is intended to be illustrative, and not restrictive. Many other implementations will be apparent to those of skill in the art upon reading and understanding the above description. Although the present disclosure has been described with reference to specific example implementations, it will be recognized that the disclosure is not limited to the implementations described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

1. A fixation element for an applicator for use in brachytherapy, the fixation element comprising:
a first component comprising a camming portion and a lever portion; and
a second component comprising a securement portion forming an opening for receiving the applicator, the securement portion comprising an abutment portion, and a first component receiving portion, wherein, in a first position, the first component is received in the first component receiving portion, and wherein the fixation element is configured to transition from the first position to a second position as the lever portion is actuated, wherein actuation of the lever portion causes the camming portion to exert a force against the abutment portion, and wherein, as the force is exerted against the abutment portion, the opening formed by the securement portion constricts.

2. The fixation element of claim 1 wherein the fixation element is resiliently deformable between the first position and the second position.

3. The fixation element of claim 1, wherein the first component further comprises at least one extending portion protruding from the camming portion, and wherein the first component receiving portion comprises a respective at least one groove arranged to receive the at least one extending portion.

4. The fixation element of claim 3, wherein the at least one extending portion is oblong.

5. The fixation element of claim 3, wherein the first component receiving portion comprises a respective at least one recess coupled to the respective at least one groove, the respective at least one recess arranged such that, in the first position, the at least one extending portion is removable from the respective at least one recess via the respective at least one groove, and in the second position, the at least one extending portion is retained in the respective at least one recess.

6. The fixation element of claim 1, wherein the second component comprises a fastening portion for securing a position of the fixation element with respect to a patient.

7. The fixation element of claim 1, wherein the second component comprises an attachment portion for securing a position of the fixation element with respect to a patient support surface.

8. The fixation element of claim 1, wherein the fixation element is configured for receiving a rectal applicator.

9. The fixation element of claim 1, wherein the camming portion is asymmetric.

10. The fixation element of claim 1, wherein the second component is resiliently compressible.

11. The fixation element of claim 1, wherein the fixation element is configured to transition from the first position to the second position through a pivoting motion of the first component.

12. The fixation element of claim 11, wherein the pivoting motion of the first component causes a rotation of the first component.

13. The fixation element of claim 12, wherein the actuation of the lever portion rotates the camming portion, the rotation of the camming portion causing the camming portion to exert a force against the abutment portion.

14. The fixation element of claim 1, wherein the fixation element is configured to transition from the first position to the second position by a deformation of the camming portion.

15. The fixation element of claim 14, wherein the deformation of the camming portion is a compression of the camming portion.

16. An applicator for brachytherapy, comprising:
a fixation element comprising:
a first component comprising a camming portion and a lever portion; and
a second component comprising a securement portion forming an opening for receiving the applicator, the securement portion comprising an abutment portion, and a first component receiving portion, wherein, in a first position, the first component is received in the first component receiving portion, and wherein the fixation element is configured to transition from the first position to a second position as the lever portion is actuated, wherein actuation of the lever portion causes the camming portion to exert a force against the abutment portion, and wherein, as the force is exerted against the abutment portion, the opening formed by the securement portion constricts.

17. The applicator of claim 16 wherein the applicator comprises a substantially cylindrical body on which the fixation element is slidably mounted when the fixation element is in the first position, and restrained on the substantially cylindrical body when in the second position.

18. The applicator of claim 17 wherein the opening is substantially circular.

19. The applicator of claim 16, wherein the applicator is a rectal applicator.
